(19) United States
(12) Patent Application Publication
(10) Pub. No.: US 2022/0161064 A1
(43) Pub. Date: May 26, 2022

(54) Filtered Air Supply Assembly

(71) Applicant: James Bledsoe, Kansas City, MO (US)

(72) Inventor: James Bledsoe, Kansas City, MO (US)

(21) Appl. No.: 17/103,474

(22) Filed: Nov. 24, 2020

(51) Int. Cl. A62B 7/10; B01D 46/00; B01D 46/44; B01D 46/42; A62B 18/02; A62B 23/02; A62B 7/12; A62B 18/00

(52) U.S. Cl. CPC A62B 7/10; B01D 46/0028; B01D 46/0023; B01D 46/0049; B01D 46/446; B01D 2279/65; A62B 18/025; A62B 23/02; A62B 7/12; A62B 18/006; B01D 46/4245

(57) ABSTRACT

A filtered air supply assembly includes a filtration unit that can be worn on a user's clothing. The filtration unit including a plurality of filters each is integrated therein and each of the filters is filter air that is pumped by the filtration unit. A mask is provided that can be worn on the user's face. A mask air hose is fluidly coupled to the mask and the mask air hose receives air from the filtration unit thereby facilitating the user to breathe the filtered air when the mask is worn. A nose air hose can be fluidly coupled to the filtration unit thereby facilitating the user to breathe the filtered air when the nose air hose is positioned in the user's nose.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to air supply devices and more particularly pertains to a new air supply device for supplying filtered breathing air.

### (2) Description of Related Art Including Information Disclosed Under 37 CFR 1.97 and 1.98

The prior art relates to air supply devices including a mask that has an integrated air filtering device. The prior art discloses a mask that has a biological filter integrated therein. The prior art discloses an air filtering device that is wearable on a user's clothing and a mask that can be worn on a user's face.

### BRIEF SUMMARY OF THE INVENTION

An embodiment of the disclosure meets the needs presented above by generally comprising a filtration unit that can be worn on a user's clothing. The filtration unit including a plurality of filters each is integrated therein and each of the filters is filter air that is pumped by the filtration unit. A mask is provided that can be worn on the user's face. A mask air hose is fluidly coupled to the mask and the mask air hose receives air from the filtration unit thereby facilitating the user to breathe the filtered air when the mask is worn. A nose air hose can be fluidly coupled to the filtration unit thereby facilitating the user to breathe the filtered air when the nose air hose is positioned in the user's nose.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a back perspective view of a filtered air supply assembly according to an embodiment of the disclosure.

FIG. 2 is an exploded perspective view of an embodiment of the disclosure.

FIG. 3 is a cross sectional view taken along line 3-3 of FIG. 1 of an embodiment of the disclosure.

FIG. 4 is a perspective view of a mask and a nose air hose an embodiment of the disclosure.

FIG. 5 is a top view of mask of an embodiment of the disclosure.

FIG. 6 is a perspective in-use view of an embodiment of the disclosure.

### DETAILED DESCRIPTION OF THE INVENTION

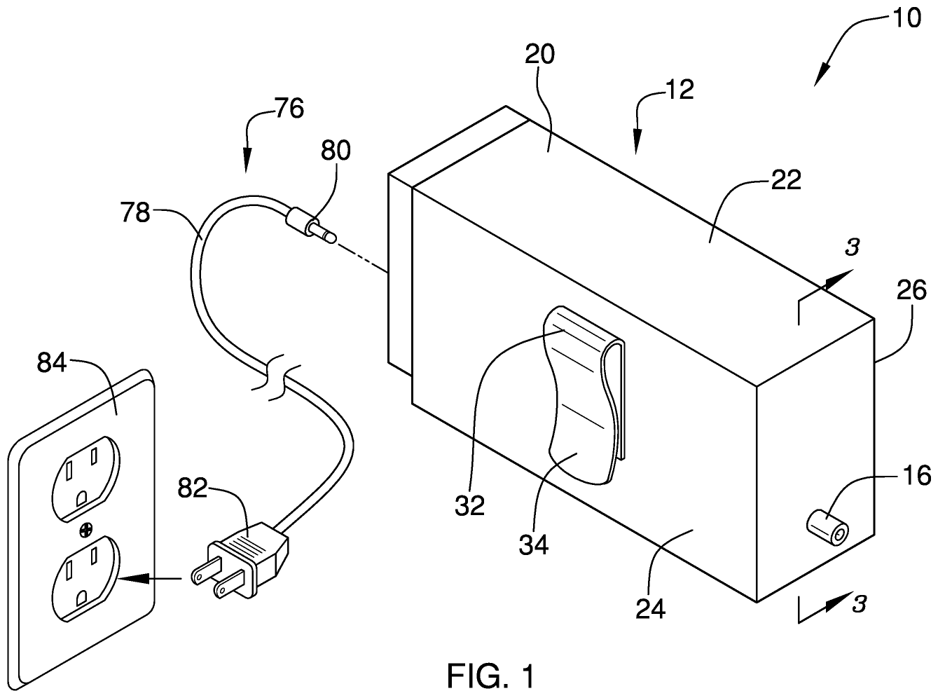

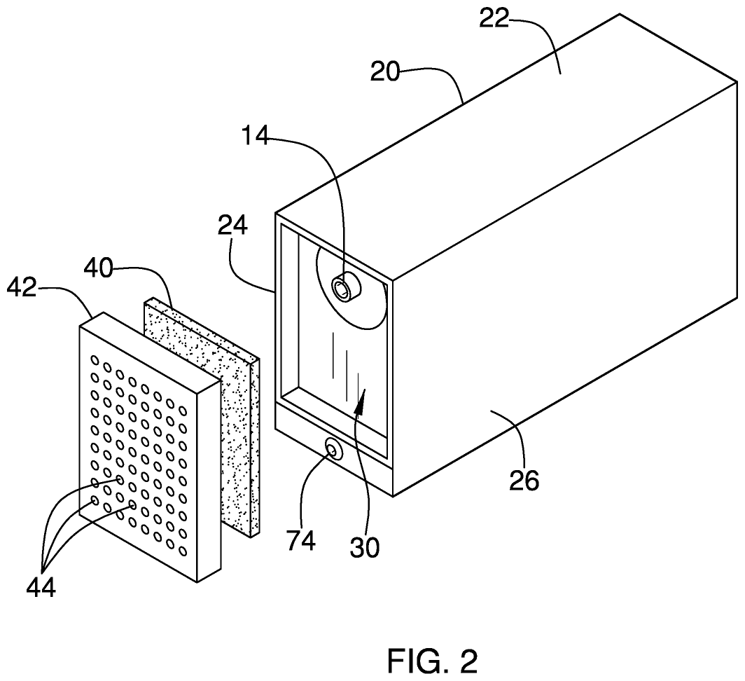

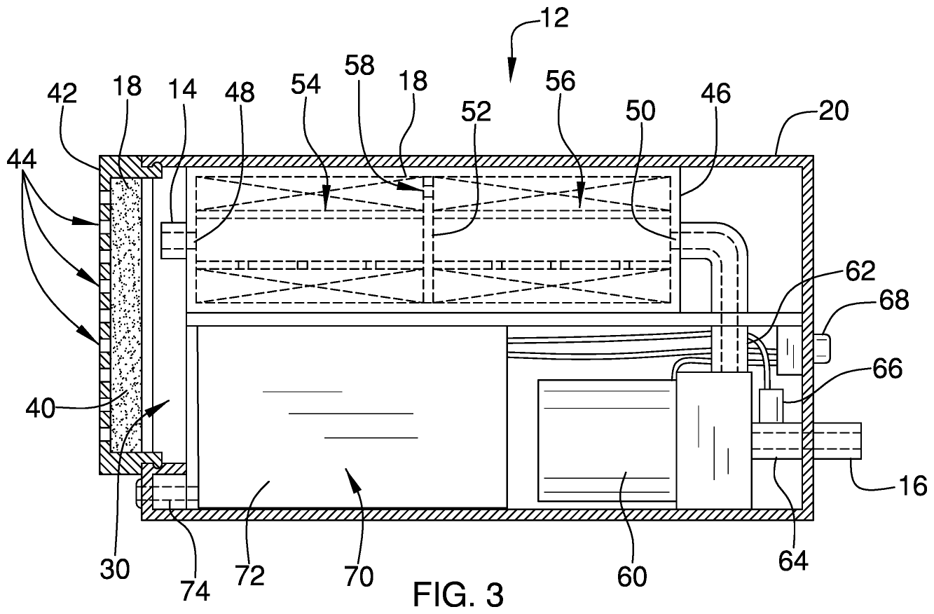

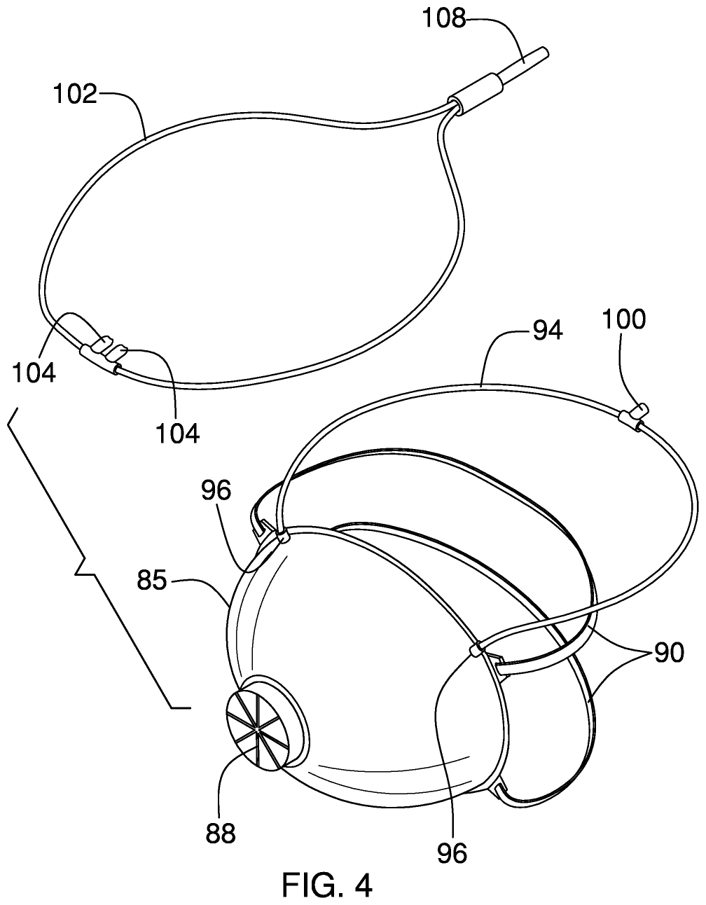

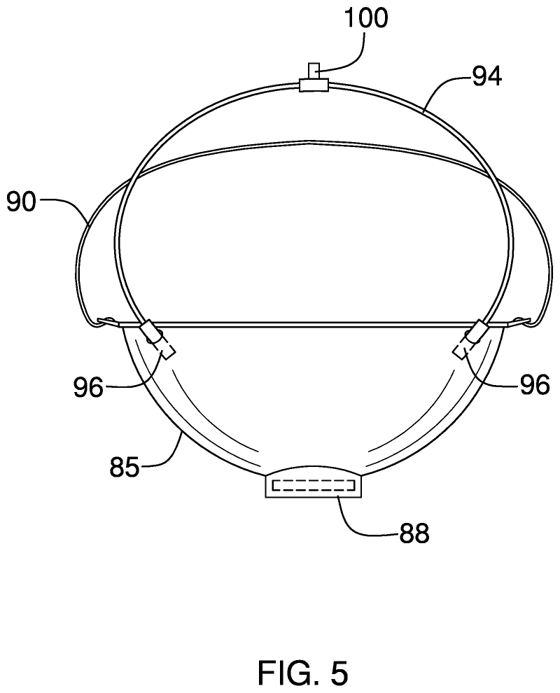

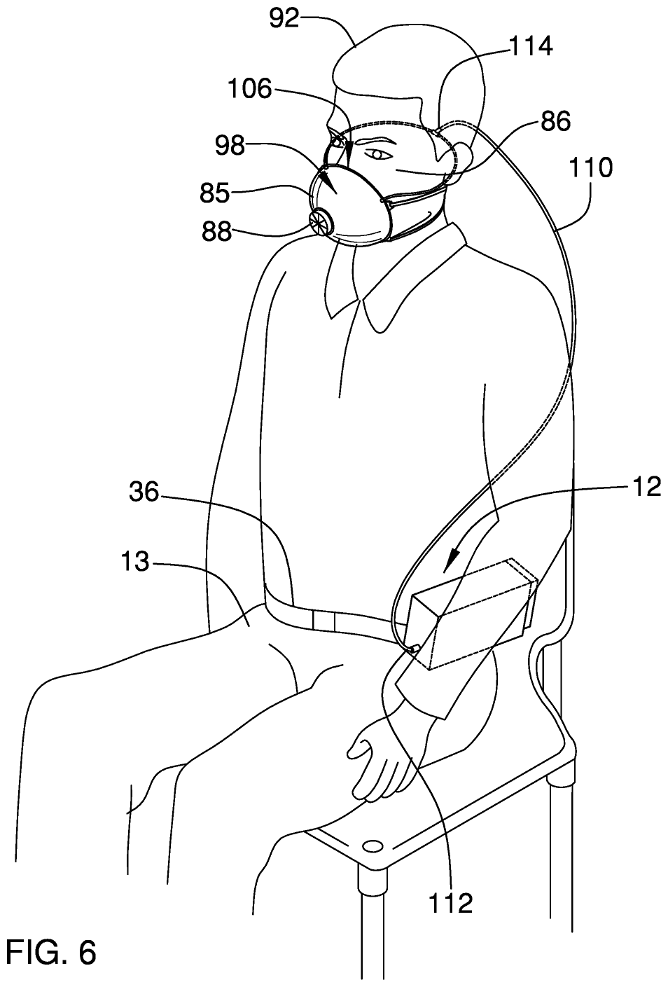

With reference now to the drawings, and in particular to FIGS. 1 through 6 thereof, a new air supply device embodying the principles and concepts of an embodiment of the disclosure and generally designated by the reference numeral 10 will be described.

As best illustrated in FIGS. 1 through 6, the filtered air supply assembly 10 generally comprises a filtration unit 12 that can be worn on a user's clothing 13. The filtration unit 12 has an intake 14 and an exhaust 16 and the filtration unit 12 urges air inwardly through the intake 14 and outwardly through the exhaust 16 when the filtration unit 12 is turned on. The filtration unit 12 includes a plurality of filters 18 that is each integrated therein to filter air urged between the intake 14 and the exhaust 16. The filtration unit 12 comprises a housing 20 has an outer wall 22 and the outer wall 22 has a first lateral side 24, a second lateral side 26 and a back side 28. The first lateral side 24 has a recess 30 extending toward the second lateral side 26. The intake 14 extends through the first lateral side 24 and the exhaust 16 extends through the second lateral side 26.

A clip 32 is coupled to the back side 28 of the outer wall 22 of the housing 20 and the clip 32 comprises a member 34 that is biased against the back side 28. The member 34 can be extended behind a waist 36 of the user's clothing 13 for mounting the housing 20 to the user's clothing 13. The plurality of filters 18 includes an intake filter 40 is removably positionable in the recess 30 and the intake filter 40 is comprised of an air permeable material to filter air urged inwardly through the intake 14. A cover 42 is removably attachable to the first lateral side 24 of the housing 20 such that the cover 42 retains the intake filter 40 in the recess 30. The cover 42 has a plurality of openings 44 each extending therethrough to pass air therethrough.

The plurality of filters 18 includes a filtration element 46 is positioned within the housing 20 and the filtration element 46 has an inlet 48 and an outlet 50. The inlet 48 is in fluid communication with the intake 14 to facilitate air to enter the filtration element 46. The filtration element 46 comprises an air permeable material to filter air passing through the filtration element 46. The filtration element 46 has a divider 52 positioned therein to define a first portion 54 of the filtration element 46 and a second portion 56 of the filtration element 46. Moreover, the divider 52 has an air hole 58 extending therethrough to pass air between the first portion 54 and the second portion 56. The filtration element 46 may comprise a HEPA filter or other type of filter that is capable of filtering not only particulates but also bacteria and viruses.

The filtration unit 12 includes a pump 60 that is positioned within the housing 20 and the pump 60 has an inlet port 62 and an outlet port 64. The pump 60 urges air inwardly through the inlet port 62 and outwardly through the outlet port 64 when the pump 60 is turned on. The inlet port 62 is fluidly coupled to the outlet 50 of the filtration element 46 such that the inlet port 62 is in fluid communication with the intake 14. In this way the pump 60 draws air through the filtration element 46 when the pump 60 is turned on. The outlet port 64 is fluidly coupled to the exhaust 16 such that the pump 60 urges filtered air outwardly through the exhaust 16. The pump 60 may comprise an electric air pump 60 or the like.

The filtration unit 12 includes a pressure sensor 66 that is coupled to the outlet port 64. The pressure sensor 66 is in fluid communication with the outlet port 64 to sense pressure of the filtered air urged outwardly through the outlet port 64. The pressure sensor 66 is electrically coupled to the pump 60 and the pressure sensor 66 turns the pump 60 off when the pressure sensor 66 senses air pressure that is below a pre-determined threshold pressure. In this way the pressure sensor 66 alerts the user to an air flow restriction.

The filtration unit 12 includes a power button 68 and the power button 68 is movably coupled to the housing 20. The power button 68 is electrically coupled to the pump 60 and the power button 68 turns the pump 60 on and off. The filtration unit 12 includes a power supply 70 that is positioned in the housing 20 and the power supply 70 is electrically coupled to the power button 68. The power supply 70 comprises at least one rechargeable battery 72 that is positioned in the housing 20 and the at least one rechargeable battery 72 is electrically coupled to the pump 60. The power supply 70 includes a charge port 74 that is integrated into the outer wall 22 of the housing 20 and the charge port 74 can be electrically coupled to a power source 76. The charge port 74 is electrically coupled to the at least one rechargeable battery 72 for charging the at least one rechargeable battery 72. The power source 76 comprises a power cord 78 that has a first plug 80 that engages the charge port 74 and a second plug 82 that engages a female electrical outlet 84.

A mask 85 is provided and the mask 85 can be worn on the user's face 86. The mask 85 has an exhaust valve 88 integrated therein to facilitate the user to exhale through the exhaust valve 88 when the mask 85 is worn. Additionally, the exhaust valve 88 inhibits air from being inhaled therethrough when mask 85 is worn. The mask 85 includes a pair of straps 90 that is each coupled thereto and each of the straps 90 can be worn around the user's head 92.

A mask air hose 94 is provided and the mask air hose 94 is fluidly coupled to the mask 85. The mask air hose 94 is positionable in fluid communication with the exhaust 16 of the filtration unit 12 to receive the filtered air from the filtration unit 12. The mask air hose 94 has a pair of outlets 96 and each of the outlets 96 extends through opposite sides of the mask 85. In this way each of the outlets 96 can direct the filtered air toward the user's mouth 98 when the mask 85 is worn. The mask air hose 94 has an input 100 that is fluidly coupled thereto.

A nose air hose 102 is provided and the nose air hose 102 is positionable in fluid communication with the exhaust 16 of the filtration unit 12 to receive the filtered air from the filtration unit 12. The nose air hose 102 has a pair of nasal ports 104 that is each fluidly coupled thereto and each of nasal ports 104 can be inserted into a respective one of the user's nostrils 106. In this way the nasal ports 104 can deliver the filtered air into the user's nostrils 106 for breathing. Additionally, the nose air hose 102 has an input 108 that is fluidly coupled thereto.

A supply hose 110 is provided and the supply hose 110 is fluidly attachable between the exhaust 16 of the filtration unit 12 and either the input 100 on the mask air hose 94 or the input 108 on the nose air hose 102. In this way the supply hose 110 can deliver the filtered air to either the mask air hose 94 or the nose air hose 102. The supply hose 110 has a first end 112 and a second end 114, and the first end 112 engages the exhaust 16 on the filtration unit 12. The second end 114 engages either the input 100 on the mask air hose 94 or the input 108 on the nose air hose 102.

In use, the first end of the supply hose 110 is fluidly coupled to the exhaust 16 of the filtration unit 12. The user wears either the mask 85 or the nose air hose 102 and the second end of the supply hose 110 is fluidly coupled to either the input on the mask air hose 94 or the input on the nose air hose 102, depending on which the user has chosen to wear. The power button 68 is depressed to turn on the pump 60 and the pump 60 begins to pump air to the supply hose 110. In this way filtered air is supplied to the user for breathing. Additionally, the filtration element 46 and the input filter inhibit the user from inhaling particulates or bacteria and viruses.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A filtered air supply assembly for supplying filtered breathing air for a user, said assembly comprising:
a filtration unit being configured to be worn on a user's clothing, said filtration unit having an intake and an exhaust wherein said filtration unit is configured to urge air inwardly through said intake and outwardly through said exhaust when said filtration unit is turned on, said filtration unit including a plurality of filters each being integrated therein wherein each of said filters is configured to filter air urged between said intake and said exhaust;
a mask being configured for wearing on the user's face;
a mask air hose being fluidly coupled to said mask, said mask air hose being positionable in fluid communication with said exhaust of said filtration unit wherein said mask air hose is configured to receive the filtered air from said filtration unit thereby facilitating the user to breathe the filtered air when said mask is worn; and
a nose air hose being positionable in fluid communication with said exhaust of said filtration unit wherein said nose air hose is configured to receive the filtered air from said filtration unit thereby facilitating the user to breathe the filtered air when said nose air hose is positioned in the user's nose.

2. The assembly according to claim 1, wherein said filtration unit comprises a housing having an outer wall, said outer wall having a first lateral side, a second lateral side and a back side, said first lateral side having a recess extending toward said second lateral side, said intake extending through said first lateral side, said exhaust extending through said second lateral side.

3. The assembly according to claim 2, wherein said filtration unit includes a clip being coupled to said back side of said outer wall of said housing, said clip comprising a member being biased against said back side wherein said member is configured to be extended behind a waist of the user's clothing for mounting said housing to the user's clothing.

4. The assembly according to claim 2, wherein said filtration unit includes an intake filter being removably positionable in said recess, said intake filter being comprised of an air permeable material wherein said intake filter is configured to filter air urged inwardly through said intake.

5. The assembly according to claim 4, wherein said filtration unit includes a cover being removably attachable to said first lateral side of said housing such that said cover retains said intake filter in said recess, said cover having a plurality of openings each extending therethrough wherein each of said openings is configured to pass air therethrough.

6. The assembly according to claim 2, further comprising a filtration element being positioned within said housing, said filtration element having an inlet and an outlet, said inlet being in fluid communication with said intake wherein said inlet is configured to facilitate air to enter said filtration element, said filtration element comprising an air permeable material wherein said filtration element is configured to filter air passing through said filtration element.

7. The assembly according to claim 6, wherein said filtration element has a divider being positioned therein to define a first portion of said filtration element and a second portion of said filtration element, said divider having an air hole extending therethrough wherein said air hole is configured to pass air between said first portion and said second portion.

8. The assembly according to claim 6, wherein said filtration unit includes a pump being positioned within said housing, said pump having an inlet port and an outlet port wherein said pump is configured to urge air inwardly through said inlet port and outwardly through said outlet port when said pump is turned on, said inlet port being fluidly coupled to said outlet of said filtration element such that said inlet port is in fluid communication with said intake wherein said pump is configured to draw air through said filtration element when said pump is turned on, said outlet port being fluidly coupled to said exhaust wherein said pump is configured to urge filtered air outwardly through said exhaust.

9. The assembly according to claim 8, wherein said filtration unit includes a pressure sensor being coupled to said outlet port, said pressure sensor being in fluid communication with said outlet port wherein said pressure sensor is configured to sense pressure of the filtered air urged outwardly through said outlet port, said pressure sensor being electrically coupled to said pump, said pressure sensor turning said pump off when said pressure sensor senses air pressure that is below a pre-determined threshold pressure wherein said pressure sensor is configured to alert a user to an air flow restriction.

10. The assembly according to claim 8, wherein said filtration unit includes a power button being movably coupled to said housing, said power button being electrically coupled to said pump, said power button turning said pump on and off.

11. The assembly according to claim 10, further comprising a power supply being positioned in said housing, said power supply being electrically coupled to said power button, said power supply comprising:
at least one rechargeable battery being positioned in said housing, said at least one rechargeable battery being electrically coupled to said pump; and
a charge port being integrated into said outer wall of said housing wherein said charge port is configured to be electrically coupled to a power source, said charge port being electrically coupled to said at least one rechargeable battery for charging said at least one rechargeable battery.

12. The assembly according to claim 1, wherein said mask air hose has a pair of outlets, each of said outlets extending through opposite sides of said mask wherein each of said outlets is configured to direct the filtered air toward the user's mouth when said mask is worn, said mask air hose having an input being fluidly coupled thereto.

13. The assembly according to claim 1, further comprising said nose air hose has a pair of nasal ports each being fluidly coupled thereto wherein each of nasal ports is configured to be inserted into a respective one of the user's nostrils thereby facilitating said nasal ports to deliver the filtered air into the user's nose for breathing, said nose air hose having an input being fluidly coupled thereto.

14. The assembly according to claim 1, wherein:
said mask air hose has an input being fluidly coupled thereto;
said nose air hose has an input being fluidly coupled thereto; and
said assembly includes a supply hose being fluid attachable between said exhaust of said filtration unit and either said input on said mask air hose or said input on said nose air hose wherein said supply hose is configured to deliver the filtered air to either said mask air hose or said nose air hose, said supply hose having a first end and a second end, said first end engaging said exhaust on said filtration unit, said second end engaging either said input on said mask air hose or said input on said nose air hose.

15. A filtered air supply assembly for supplying filtered breathing air for a user, said assembly comprising:
a filtration unit being configured to be worn on a user's clothing, said filtration unit having an intake and an exhaust wherein said filtration unit is configured to urge air inwardly through said intake and outwardly through said exhaust when said filtration unit is turned on, said filtration unit including a plurality of filters each being integrated therein wherein each of said filters is configured to filter air urged between said intake and said exhaust, said filtration unit comprising:
a housing having an outer wall, said outer wall having a first lateral side, a second lateral side and a back side, said first lateral side having a recess extending toward said second lateral side, said intake extending through said first lateral side, said exhaust extending through said second lateral side;
a clip being coupled to said back side of said outer wall of said housing, said clip comprising a member being biased against said back side wherein said member is configured to be extended behind a waist of the user's clothing for mounting said housing to the user's clothing;
an intake filter being removably positionable in said recess, said intake filter being comprised of an air permeable material wherein said intake filter is configured to filter air urged inwardly through said intake;
a cover being removably attachable to said first lateral side of said housing such that said cover retains said intake filter in said recess, said cover having a plurality of openings each extending therethrough wherein each of said openings is configured to pass air therethrough;
a filtration element being positioned within said housing, said filtration element having an inlet and an outlet, said inlet being in fluid communication with said intake wherein said inlet is configured to facilitate air to enter said filtration element, said filtration element comprising an air permeable material wherein said filtration element is configured to filter air passing through said filtration element, said filtration element having a divider being positioned therein to define a first portion of said filtration element and a second portion of said filtration element, said divider having an air hole extending therethrough wherein said air hole is configured to pass air between said first portion and said second portion;
a pump being positioned within said housing, said pump having an inlet port and an outlet port wherein said pump is configured to urge air inwardly through said inlet port and outwardly through said outlet port when said pump is turned on, said inlet port being fluidly coupled to said outlet of said filtration element such that said inlet port is in fluid communication with said intake wherein said pump is configured to draw air through said filtration element when said pump is turned on, said outlet port being fluidly coupled to said exhaust wherein said pump is configured to urge filtered air outwardly through said exhaust;
a pressure sensor being coupled to said outlet port, said pressure sensor being in fluid communication with said outlet port wherein said pressure sensor is configured to sense pressure of the filtered air urged outwardly through said outlet port, said pressure sensor being electrically coupled to said pump, said pressure sensor turning said pump off when said pressure sensor senses air pressure that is below a pre-determined threshold pressure wherein said pressure sensor is configured to alert a user to an air flow restriction;
a power button being movably coupled to said housing, said power button being electrically coupled to said pump, said power button turning said pump on and off; and
a power supply being positioned in said housing, said power supply being electrically coupled to said power button, said power supply comprising:
at least one rechargeable battery being positioned in said housing, said at least one rechargeable battery being electrically coupled to said pump; and
a charge port being integrated into said outer wall of said housing wherein said charge port is configured to be electrically coupled to a power source, said charge port being electrically coupled to said at least one rechargeable battery for charging said at least one rechargeable battery;
a mask being configured for wearing on the user's face, said mask having an exhaust valve being integrated therein wherein said exhaust valve is configured to facilitate the user to exhale through said exhaust valve when said mask is worn, said mask including a pair of straps each being coupled thereto wherein each of said straps is configured to be worn around the user's head;
a mask air hose being fluidly coupled to said mask, said mask air hose being positionable in fluid communication with said exhaust of said filtration unit wherein said mask air hose is configured to receive the filtered air from said filtration unit, said mask air hose having a pair of outlets, each of said outlets extending through opposite sides of said mask wherein each of said outlets is configured to direct the filtered air toward the user's mouth when said mask is worn, said mask air hose having an input being fluidly coupled thereto;
a nose air hose being positionable in fluid communication with said exhaust of said filtration unit wherein said nose air hose is configured to receive the filtered air from said filtration unit, said nose air hose having a pair of nasal ports each being fluidly coupled thereto wherein each of nasal ports is configured to be inserted into a respective one of the user's nostrils thereby facilitating said nasal ports to deliver the filtered air into the user's nose for breathing, said nose air hose having an input being fluidly coupled thereto; and
a supply hose being fluid attachable between said exhaust of said filtration unit and either said input on said mask air hose or said input on said nose air hose wherein said supply hose is configured to deliver the filtered air to either said mask air hose or said nose air hose, said supply hose having a first end and a second end, said first end engaging said exhaust on said filtration unit, said second end engaging either said input on said mask air hose or said input on said nose air hose.
